(19) United States
(12) Patent Application Publication
(10) Pub. No.: US 2023/0115239 A1
(43) Pub. Date: Apr. 13, 2023

(54) BIOCOMPATIBLE SCAFFOLD AND USE THEREOF

(71) Applicant: TECHNION RESEARCH & DEVELOPMENT FOUNDATION LIMITED, Haifa (IL)

(72) Inventors: Shulamit LEVENBERG, Haifa (IL); Havazelet BIANCO-PELED, Haifa (IL); Majd MACHOUR, Haifa (IL); Noy HEN, Haifa (IL)

(21) Appl. No.: 17/960,972

(22) Filed: Oct. 6, 2022

Related U.S. Application Data
(60) Provisional application No. 63/253,173, filed on Oct. 7, 2021.

(51) Int. Cl. A61L 27/52; A61L 27/20; A61L 27/22; A61L 27/50; C12M 1/12

(52) U.S. Cl. CPC A61L 27/52; A61L 27/20; A61L 27/22; A61L 27/507; C12M 25/14

(57) ABSTRACT

A kappa-carrageenan (Kcar) granular hydrogel devoid of a cell-toxic crosslinking agent is provided as a scaffold for maintaining and implanting cellular structures such as lumens. The lumens may be defined by cells or surrounded by cells and may have the dimensions of a blood vessel.

### CROSS REFERENCE TO RELATED APPLICATIONS

This application claims the benefit of priority of U.S. Provisional Patent Application No. 63/253,173 filed Oct. 7, 2021, entitled “BIOCOMPATIBLE SCAFFOLD AND USE THEREOF”, the contents of which are incorporated herein by reference in their entirety.

### FIELD OF INVENTION

The present invention, in some embodiments thereof, is directed to 3D scaffolds with structural and functional properties that mimic the 3D environment of living tissue.

### BACKGROUND OF THE INVENTION

A major challenge in tissue engineering is to fabricate 3D scaffolds with structural and functional properties that mimic the 3D environment of living tissue. Recently, 3D bioprinting has gained wide interest for its potential to create such scaffolds. Specifically, extrusion based bioprinting emerged as a useful technique to fabricate cellular 3D scaffolds with high complexity and accuracy. In this technique, a wide range of biomaterials called bioinks can be incorporated with living cells and extruded through a nozzle in a layer-by-layer manner. Following extrusion, the bioinks undergo gelation through diverse cross-linking mechanisms, either during or after the printing process.

An inherent limitation of extrusion printing onto a hard surface is that it requires high viscosity biomaterials to prevent deformation and collapse of the delicate structure. On the other hand, increasing the viscosity can damage cellular function and cell migration. To overcome this obstacle, a method of freeform direct printing into a granular support material was developed.

Support materials are yield stress fluids with shear thinning and self-healing properties, composed of suspended particles. They liquefy under high shear stress but behave as a rigid matrix under low shear stress. Thus, the needle moves freely during printing, whereas once the stress is removed the extruded bioink is trapped between the particles and maintains its structure. The printed structure is then cured, followed by removal of the support material using an external trigger such as temperature change, enzymatic cleavage or mechanical force. Finally, the printed construct is transferred into a liquid medium for tissue maturation and growth.

Direct printing within support material provides mechanical support to the bioink and is therefore suitable for low viscosity biomaterials that is compatible with cells. This technique has been successfully applied while utilizing various types of support materials including polyacrylic acid microgels, gelatin microparticles, agarose, and alginate combined with xanthan gum gels.

However, a common phenomenon observed in 3D cellular hydrogels, such as those that are generated using 3D bioprinting, is the contraction of the gel mediated by cellular traction forces. This contraction results from insufficient strength of the hydrogel network fibers that buckles under the force of the cells acting on them.

Hydrogel contraction reduces the volume of the construct and presents several problems. First, for applications in tissue replacement, the design of the construct is based on patient-specific imaging, which the implanted engineered tissue needs to accommodate. The contraction of the tissue presents a major hurdle to allow for the implantation of the tissue. Second, the volume reduction leads to an increase of cell density within the construct, as well as the hydrogel network density and porosity. Since these variables play an important role in cell viability, differentiation state and function, the contraction of the hydrogel might lead to undesired effects on the engineered tissue. Furthermore, the degree of contraction might vary depending on cell type, cell passage, and variability in the batch of cells or hydrogel materials.

Despite this, most works report the initial geometry and cell density of the hydrogel, without reporting on the final density or the final construct volume for the experiment time points. This makes reproducing the same results from different similar experiments difficult and unpredictable.

A few methods have been suggested in previous works that aim to counteract the contraction phenomenon or make use of it to achieve a specific desired effect. For example, some works describe using a synthetic polymer ink such as PCL to print a supporting structure for the cellular hydrogel bioink. However, these supporting structures are often a permanent constituent of the tissue prolonging its in vivo degradation time. Moreover, this method often requires using high-temperature extrusion of the polymer ink next to cells which might lead to reduced viability. In other examples, researchers used an anchoring type of support structures, where the contraction in limited in one direction. This led to a directional contraction of the structure which aided the development of aligned muscle fibers. However, this type of support is very limiting, especially for translating the method for larger and more complex tissue applications. Lastly, the contraction of a bioprinted cellular collagen-methacrylate structure was used to fuse the construct to a separately fabricated PLLA-PLGA scaffold. Thus, a more uniform method of counteracting contraction of bioprinted constructs is needed, where the support is external and removable, without affecting the viability and function of the cells.

### SUMMARY OF THE INVENTION

In one embodiment, provided a kappa-carrageenan (Kcar) granular hydrogel devoid of a cell-toxic crosslinking agent.

In another embodiment, provided a water in oil emulsion comprising the kappa-carrageenan (Kcar) granular hydrogel.

In another embodiment, provided a composition comprising the water in oil emulsion comprising the kappa-carrageenan (Kcar) granular hydrogel, a lipophilic non-ionic surfactant, and an ionic cross linker. In another embodiment, provided a composition comprising the water in oil emulsion comprising soluble kappa-carrageenan (Kcar. In another embodiment, provided a composition comprising the water in oil emulsion comprising soluble kappa-carrageenan (Kcar), a lipophilic non-ionic surfactant, and an ionic cross linker.

In another embodiment, provided a composition comprising a kappa-carrageenan (Kcar) granular hydrogel and potassium chloride. In another embodiment, provided a composition comprising a kappa-carrageenan (Kcar) granular hydrogel and potassium chloride and lipophilic non-ionic surfactant.

In another embodiment, provided a composition comprising a kappa-carrageenan (Kcar) granular hydrogel and a bioink.

In another embodiment, provided a composition comprising a kappa-carrageenan (Kcar) granular hydrogel and potassium chloride.

In another embodiment, provided a particle comprising a kappa-carrageenan (Kcar) granular hydrogel, characterized by a diameter of 1 micrometer to 100 micrometers.

In another embodiment, provided a composition comprising a kappa-carrageenan (Kcar) granular hydrogel encapsulating a cell.

In another embodiment, provided a composition comprising a kappa-carrageenan (Kcar) granular hydrogel, and at least one compound selected from: calcium chloride, thrombin, fibrin, gelatin-methacryloyl, or any combination thereof.

In another embodiment, provided a composition comprising a kappa-carrageenan (Kcar) granular hydrogel and cell culture media.

In another embodiment, provided a composition comprising a kappa-carrageenan (Kcar) granular hydrogel, wherein the hydrogel comprises a lumen within its matrix. In another embodiment, the lumen is having the structure and dimensions of a blood vessel. In another embodiment, the lumen is defined or surrounded by cells.

In another embodiment, provided a method for fabricating a cell carrier, comprising inserting a cell into a composition comprising the kappa-carrageenan (Kcar) granular hydrogel.

In another embodiment, provided a method for implanting a cell to a subject, comprising administering to the subject the composition the kappa-carrageenan (Kcar) granular hydrogel encapsulating a cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1i-ii: Is an illustration of the procedure of the invention. (i); microgels dispersed in a fluid or sediment into a microporous granular hydrogel; (ii) bioprinting and formation of a vessel.

FIG. 2A-2I: Microgels characterization: (A) light microscopy image of dilute microgels and (B) cryo-SEM image of the sedimented microgels within the continuous phase (KCl). (C) size distribution of the microgels (n=6). Representative results of rheological characterization using (D) strain sweep test (E) temperature sweep test (F) steady shear flow test and (G) thixotropic test, indicating on shear-thinning and self-healing behavior. (H) images of the granular hydrogel (with cover slip) and gelatin support bath to demonstrate the transparency of the microgels. (I) the diffusion coefficients of fluorescein tracer through KCar microgels, gelatin support bath and PBS (n=6). scale bars: (A) 50 μm (B) 20 μm (H) 10 μm (student t-test).

FIG. 3A-3D: Depicts 3D printing within the granular hydrogel: (A) A computer design of rectilinear pattern (4 layers) and representative images after printing of alginate, GelMA and fibrin bioinks within the granular hydrogel. (B) A computer design of hollow diverging vessel and representative images after extraction of alginate, GelMA and fibrin bioinks. (C) A computer design of the university logo and representative images after printing of alginate and fibrin within the granular hydrogel and extracted GelMA. (D) A semi-quantitative evaluation of the geometric accuracy and compression between the bioinks (n=3). Scale bar: (A) 1 mm (B-C) 2 mm.

FIG. 4A-4C: Incubation in granular hydrogel reduced cellular construct contraction: (A) fluorescence images of printed HNDF constructs within liquid medium or granular hydrogel during culture. (B) quantification of the normalized printed area during 48 hours of culture (n=4, P<0.0005). (C) Images of a hollow diverging vessel structure containing HNDF cells. The printed constructs incubated within liquid medium (right) or granular hydrogel (left) for 8 days culture. Scale bar (A) 2 mm (C) 5 mm.

FIG. 5A-5G: Depicts cell viability and proliferation during culture in the granular hydrogel. (A) 100 micrometer (B) 100 micrometer (C) N=7 (D) N=5 (E) N=8 (F) 100 micrometer) (G) N=6.

FIG. 6A-6E: Depicts Immunofluorescent staining for nuclear early osteogenic marker RUNX and late cytoplasmic osteogenic marker BSP-2 show similar expression in both groups (100 micrometer)(A), alizarin red assay (B, N=3)) and Young modulus results (C, N=3), functional assay for myoblasts is provide (D. N=11) and isoproterenol/baseline assay (E).

### DETAILED DESCRIPTION OF THE INVENTION

The present invention, in some embodiments thereof, is directed to compositions and methods in 3D printing into a granular support material aimed to enhance the long-term structural stability of the printed objects.

In one embodiment, provided herein is a hydrogel-based composition for culturing cells and maintaining their viability. In one embodiment, provided herein is a hydrogel-based composition capable of receiving a cell injected therein. In one embodiment, provided herein is a hydrogel-based composition encapsulating a cell.

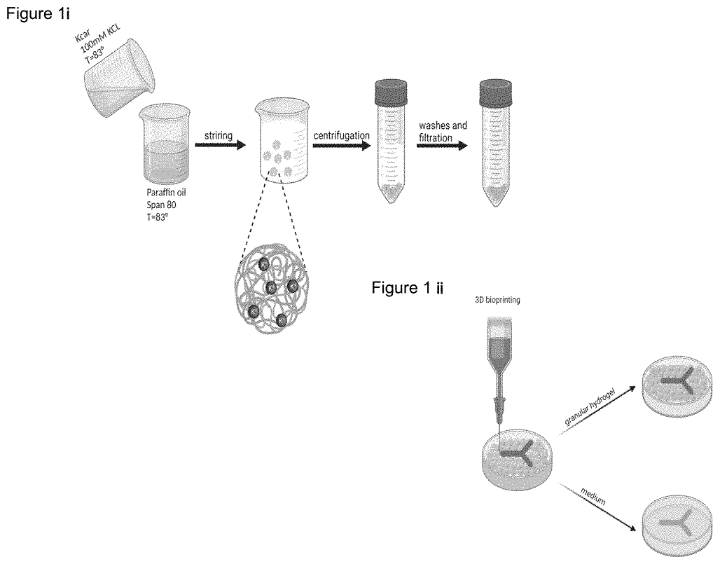

In one embodiment, in the present method the printed construct such as a cell or a living tissue is not removed from the support material (the hydrogel) immediately after insert (printing), but rather is incubated within it for prolong times (see FIG. 1). In one embodiment, the support material comprises a kappa-carrageenan microgel.

In one embodiment, the present method enables long-term mechanical support to the printed construct (a cell or a living tissue), limit its contraction, and thus enable printing of complex cellular structures from a low viscosity biomaterial that provides a cell-friendly environment. In one embodiment, the present method enables direct live-cell imaging during culture.

In one embodiment, the present invention provides fabrication of a new granular material based on a kappa-carrageenan microgel which is suitable for encapsulating, housing, maintaining, printing, growing, differentiating, propagating, or any combination thereof of a cell or a tissue. In one embodiment, the kappa-carrageenan microgel of the invention enables in-situ characterization of cells encapsulated therein. In one embodiment, the kappa-carrageenan microgel supports cell culture over time 1, 2, 3, 4, 5, 6, 7, 8, 9, 10, 11, 12, 13, 14, 15, 17, 21, 25, 28, 35, 40, 45 day/s or any value therebetween.

In one embodiment, a composition as described herein comprises a kappa-carrageenan (Kcar) microgel. In one embodiment, Kcar microgel is fabricated using water in oil (W/O) emulsion. In one embodiment, a microgels as described herein is a polymeric microscale particle with high water content (60-99.8% by weight) and porosity. In one embodiment, a microgels as described herein is a porous polymeric microscale particle with a water content 70-99.8% by weight.

In one embodiment, Kcar comprises the structure:

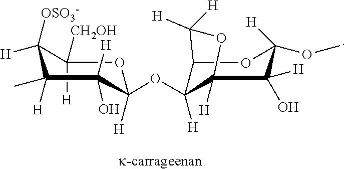

In one embodiment, provided herein a kappa-carrageenan (Kcar) granular hydrogel devoid of a cell-toxic crosslinking agent. In one embodiment, provided herein a composition comprising kappa-carrageenan (Kcar) granular hydrogel wherein the composition is devoid of a cell-toxic crosslinking agent. In one embodiment, provided herein a composition comprising a cell and a kappa-carrageenan (Kcar) granular hydrogel wherein the composition is devoid of a cell-toxic crosslinking agent. In one embodiment, provided herein a composition comprising a cell culture and a kappa-carrageenan (Kcar) granular hydrogel wherein the composition is devoid of a cell-toxic crosslinking agent. In one embodiment, provided herein a composition comprising a tissue culture and a kappa-carrageenan (Kcar) granular hydrogel wherein the composition is devoid of a cell-toxic crosslinking agent. In one embodiment, a composition as described here is devoid of any cytotoxic compound/agent. In one embodiment, a kappa-carrageenan (Kcar) granular hydrogel as described here is devoid of any cytotoxic compound/agent. In one embodiment, provided herein a composition comprising an organ or a tissue derived from an organ and a kappa-carrageenan (Kcar) granular hydrogel wherein the composition is devoid of a cell-toxic crosslinking agent.

In one embodiment, Kappa carrageenan (Kcar) hydrogel is water based. In one embodiment, Kappa carrageenan (Kcar) hydrogel comprises 70-99% w/w water. In one embodiment, Kappa carrageenan (Kcar) hydrogel comprises 75-99% w/w water. In one embodiment, Kappa carrageenan (Kcar) hydrogel comprises 85-99% w/w water. In one embodiment, Kappa carrageenan (Kcar) hydrogel comprises 70-99% w/v water. In one embodiment, Kappa carrageenan (Kcar) hydrogel comprises 75-99% w/v water. In one embodiment, Kappa carrageenan (Kcar) hydrogel comprises 85-99% w/v water. In one embodiment, Kappa carrageenan (Kcar) hydrogel comprises 0.001 to 2% (w/v) Kappa carrageenan. In one embodiment, Kappa carrageenan (Kcar) hydrogel comprises 0.01 to 0.5% (w/v) Kappa carrageenan. In one embodiment, Kappa carrageenan (Kcar) hydrogel comprises 0.01 to 0.1% (w/v) Kappa carrageenan. In one embodiment, Kappa carrageenan (Kcar) hydrogel comprises 0.01 to 0.08% (w/v) Kappa carrageenan. In one embodiment, Kappa carrageenan (Kcar) hydrogel comprises 0.01 to 0.05% (w/v) Kappa carrageenan. In one embodiment, Kappa carrageenan (Kcar) hydrogel comprises 0.017 to 0.1% (w/v) Kappa carrageenan. In one embodiment, Kappa carrageenan (Kcar) hydrogel comprises 1.5% (w/v) Kappa carrageenan (Sigma Aldrich) and 90 ml solution of 100 mM potassium chloride (KCl) in water. In one embodiment, Kappa carrageenan (Kcar) hydrogel comprises 0.5-2.5% (w/v) Kappa carrageenan (Sigma Aldrich) and 70-120 90 ml water solution of 50-150 mM potassium chloride (KCl). In one embodiment, Kappa carrageenan (Kcar) hydrogel comprises 0.0002-0.8% (v/v) lipophilic surfactant. In one embodiment, Kappa carrageenan (Kcar) hydrogel comprises 0.0002-0.008% (v/v) lipophilic surfactant. In one embodiment, Kappa carrageenan (Kcar) hydrogel comprises 0.05-0.8% (v/v) lipophilic surfactant. In one embodiment, Kappa carrageenan (Kcar) hydrogel comprises 0.05-0.4% (v/v) lipophilic surfactant. In one embodiment, Kappa carrageenan (Kcar) hydrogel comprises 0.1-0.3% (v/v) lipophilic surfactant. In one embodiment, the lipophilic surfactant comprises span80.

In one embodiment, the phrase cell-toxic includes cytotoxic. In one embodiment, cytotoxic effect compromise cell membrane integrity. In one embodiment, cytotoxic effect induces cell death. In one embodiment, cytotoxic effect induces cell shrinkage. In one embodiment, cytotoxic effect inhibits cell proliferation. In one embodiment, cytotoxic effect inhibits cell metabolism. In one embodiment, cytotoxic effect induces lethal redox potential of cells.

In one embodiment, provided herein a water in oil emulsion comprising the kappa-carrageenan (Kcar) granular hydrogel. In one embodiment, provided herein a water in oil emulsion comprising a soluble kappa-carrageenan (Kcar). In one embodiment, provided herein a water in oil emulsion comprising a soluble kappa-carrageenan (Kcar) and a lipophilic non-ionic surfactant. In one embodiment, provided herein a water in oil emulsion comprising a soluble kappa-carrageenan (Kcar) and an ionic cross linker. In one embodiment, provided herein a composition comprising a kappa-carrageenan (Kcar) granular hydrogel and an ionic cross linker. In one embodiment, provided herein a composition comprising a kappa-carrageenan (Kcar) granular hydrogel and a lipophilic non-ionic surfactant.

In another embodiment, provided a composition comprising the water in oil emulsion, a lipophilic non-ionic surfactant, and an ionic cross linker. In another embodiment, provided a composition comprising the water in oil emulsion, a lipophilic non-ionic surfactant, a cell and an ionic cross linker.

In one embodiment, provided herein a water in oil emulsion comprising a soluble kappa-carrageenan (Kcar) and potassium chloride. In one embodiment, provided herein a composition comprising a kappa-carrageenan (Kcar) granular hydrogel and potassium chloride.

In one embodiment, a composition as described herein further comprises a bioink. In one embodiment, a kappa-carrageenan (Kcar) granular hydrogel comprises a bioink.

In one embodiment, provided herein is a particle comprising a kappa-carrageenan (Kcar) granular hydrogel. In one embodiment, the particle comprising is characterized by a diameter of 1 micrometer to 100 micrometers. In one embodiment, the particle comprising is characterized by a diameter of 5 micrometer to 100 micrometers. In one embodiment, the particle comprising is characterized by a diameter of 5 micrometer to 80 micrometers. In one embodiment, the particle comprising is characterized by a diameter of 10 micrometer to 60 micrometers. In one embodiment, the particle comprising is characterized by a diameter of 10 micrometer to 40 micrometers. In one embodiment, the particle comprising is characterized by a diameter of 15 micrometer to 30 micrometers.

In one embodiment, the particle comprises a viable cell. In one embodiment, the particle comprises a viable stained cell. In one embodiment, the particle comprises a cell culture media. In one embodiment, the particle comprises a cell culture media and a viable cell.

In one embodiment, a composition comprising a kappa-carrageenan (Kcar) granular hydrogel encapsulates a cell. In one embodiment, a composition comprising a kappa-carrageenan (Kcar) granular hydrogel encapsulates a cell and a bioink. In one embodiment, a kappa-carrageenan (Kcar) granular hydrogel comprises a lumen or a channel. In one embodiment, a lumen or a channel is a void. In one embodiment, a lumen or a channel is surrounded by cells. In one embodiment, the matrix of a kappa-carrageenan (Kcar) granular hydrogel comprises at least one lumen. In one embodiment, the lumen is characterized by having the structure and dimensions of a blood vessel.

In one embodiment, a composition as described herein further comprises a growth factor or a cell attachment molecule. In one embodiment, a composition as described herein further comprises calcium chloride. In one embodiment, a composition as described herein further comprises thrombin. In one embodiment, a composition as described herein further comprises fibrin. In one embodiment, a composition as described herein further comprises gelatin-methacryloyl. In one embodiment, a composition as described herein further comprises a bodily fluid.

In one embodiment, provided herein is a method for fabricating a cell carrier, comprising inserting, injecting and/or printing a cell into a composition comprising the kappa-carrageenan (Kcar) granular hydrogel. In one embodiment, provided herein is a method for fabricating a cell carrier, comprising inserting, injecting and/or printing a eukaryotic cell into a composition comprising the kappa-carrageenan (Kcar) granular hydrogel. In one embodiment, provided herein is a method for fabricating a cell carrier, comprising inserting, injecting and/or printing a bacterial cell into a composition comprising the kappa-carrageenan (Kcar) granular hydrogel.

In one embodiment, provided herein is a method for implanting a cell to a subject, comprising administering to the subject the composition comprising the kappa-carrageenan (Kcar) granular hydrogel, wherein the Kcar granular hydrogel comprises the cell. In one embodiment, provided herein is a method for implanting a cell to a subject, comprising administering to the subject the composition comprising the kappa-carrageenan (Kcar) granular hydrogel encapsulating the cell. In one embodiment, provided herein is a method for implanting a cell to a subject comprises cell therapy. In one embodiment, provided herein is a method for implanting a cell to a subject comprises cell replacement therapy.

In one embodiment, KCar is a biocompatible polysaccharide originating from red seaweeds. In one embodiment, KCar is a thermosensitive polymer that undergoes gelation during cooling or in the presence of monovalent cations.

In one embodiment, the gelation process involves helix formation followed by further aggregations to form a strong transparent hydrogel. In one embodiment, provided herein is a stable KCar microgel devoid of any toxic crosslinking agent.

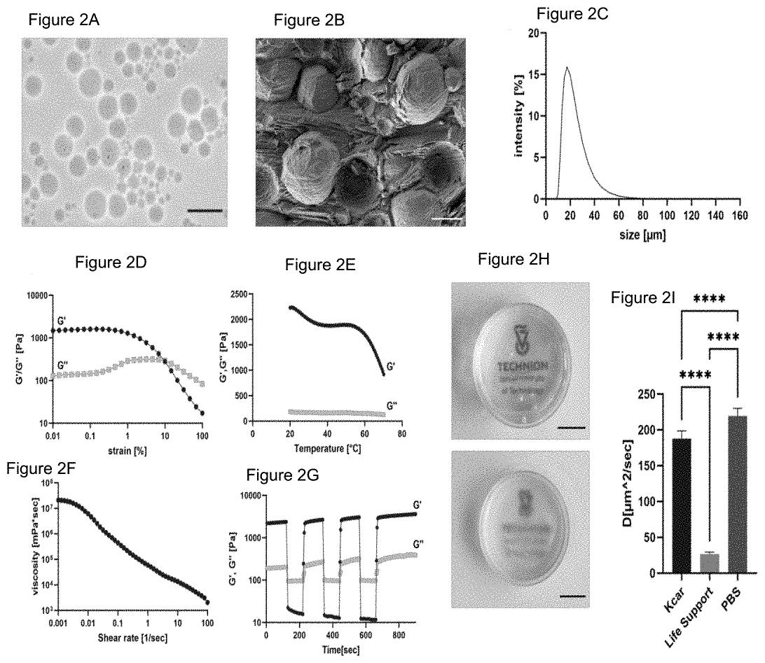

In one embodiment, the process for preparing a microgel includes creating a water in oil (W/O) emulsion at high temperature (see FIG. 2E, above 40° C. and below 80° C., above 50° C. and below 80° C. or above 60° C. and below 80° C.), followed by cooling the emulsion to induce gelation of KCar and separation of the microgels (below 40° C. and above 10° C., below 30° C. and above 10° C., or below 25° C. and above 10° C.).

In one embodiment, an aqueous precursor solution comprising KCar and ionic cross linker (potassium chloride, KCl) was mixed with an oil phase at high temperature in which the polymer is soluble and stirred to generate water droplets within the continuous oil phase. In one embodiment, the polymeric water droplets were stabilized using a lipophilic surfactant such as but not limited to span®80. In one embodiment, the lipophilic surfactant is also non-ionic.

In one embodiment, upon cooling to room temperature the hydrogel of the invention cures and solidifies, which allowed it separation using centrifugation. In one embodiment, the spherical morphology of the obtained microgels is visible in bright field microscopy images (FIG. 2A) and cryo-SEM micrographs (FIG. 2B).

In one embodiment, the size distribution of the microgels indicated that small particles with number average diameter of 2-40 micrometers, 5-40 micrometers, 10-40 micrometers, 20-40 micrometers, 10-30 micrometers, or 21.4 μm±2.1 μm are obtained (FIG. 2C).

In one embodiment, the granular hydrogel has a gel-like behavior (G′>G″) in all tested temperatures, particularly at ambient temperature and at physiological temperature (10-50° C.). In one embodiment, this behavior enables continuous mechanical support of solid particles during printing and cellular construct incubation. The shear-thinning behavior was studied using steady shear flow measurements (FIG. 2F). In one embodiment, the granular hydrogel exhibits a decrease in viscosity with an increase of shear rate which was well fitted to simple power low model derived from the Carreau model: η=k×\dot{γ}ⁿ⁻¹; where η is the measured viscosity, γ is the shear rate, k is the consistency coefficient and n is the power low index or flow behavior index. The power law index depends on the shear thinning characteristics of a fluid; n<1 for shear thinning materials whereas n>1 for shear thickening materials.

In one embodiment, the granular hydrogel exhibits a gel-like behavior under low shear strains (G′>G″) and a liquid-like behavior under high shear strains (G″>G′). In one embodiment, the granular hydrogel exhibits rapid and full recovery in all cycles indicating the self-healing properties of the granular hydrogel. In one embodiment, the granular hydrogel is transparent.

In one embodiment, a bioink-an organic or biological ink is used as a printing ink into the granular hydrogel. In one embodiment, a bioink is selected from: Alginate with calcium chloride as ionic cross linker, gelatin-methacryloyl (GelMA) that undergoes photo-cross linking and fibrin which undergoes enzymatic cross-linking.

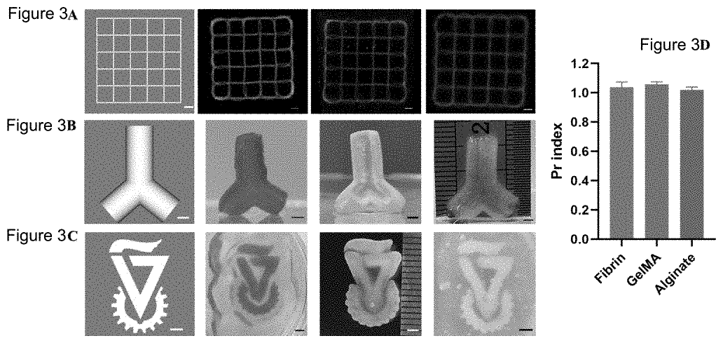

In one embodiment, a bioink further comprises a visualization aid compound such as alcian blue powder or fluorescent microspheres (FIG. 3A). In one embodiment, a semi-quantitative evaluation of the printed structures using the printability index (Pr) to quantify geometric accuracy within the granular hydrogel (FIG. 3D). In one embodiment, the printed granular hydrogel displays high geometric accuracy compared to the designed model and lack of significant difference between the bioinks, demonstrating the versatility of the granular hydrogel.

In one embodiment, a lumen is created/printed within the granular hydrogel. In one embodiment, an open lumen is created/printed within the granular hydrogel. In one embodiment, a perfusable lumen is created/printed within the granular hydrogel. In one embodiment, a hollow diverging vessel having the dimension of a blood vessel was printed inside the granular hydrogel using the same bioinks and extracted from the granular hydrogel (FIG. 3B).

In one embodiment, a cell is printed/injected in or with and/or on a hydrogel as described herein. In one embodiment, a cell is caged or encapsulated within a hydrogel as described herein. In one embodiment, a cell is a eukaryotic cell. In one embodiment, a cell is a bacterial cell. In one embodiment, a cell is a mesenchymal cell or MSC. In one embodiment, a cell is a labelled cell. In one embodiment, a labelled cell a fluorophore or a chromophore. In one embodiment, a labelled cell is radioactively labelled. In one embodiment, a cell or a labelled cell is encapsulated in a fibrin-based bioink. In one embodiment, a cell is printed in a concentric disc shape with an outer and inner diameter of 7 mm and 3 mm respectively. In one embodiment, a printed or printing is encapsulating a cell as described herein.

In one embodiment, a hydrogel scaffold comprising or encapsulating a cell as described herein is further contacted with a KCar support medium. In one embodiment, a hydrogel scaffold comprising or encapsulating a cell such as MSCs result in a vessel structure composed of cells/MSCs.

In one embodiment, a hydrogel scaffold provides superior support for an encapsulated cell or a multi-cell structure such a vessel. In one embodiment, a hydrogel scaffold as described herein minimizes scaffold contraction during culture. In one embodiment, the present hydrogel scaffold is based upon the formation of ionic cross link microgels using W/O emulsion ensure desire morphology and does not involve toxic reagents. In one embodiment, common bioinks are printed in the hydrogel with different cross linkers inside the granular hydrogel.

### General

As used herein the term “about” refers to ±10%. The terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### EXAMPLES

### Materials and Methods

### Microgels Preparation

Kappa carrageenan (Kcar) microgels were prepared using W/O emulsion technique. 1.5% (w/v) Kappa carrageenan (Sigma Aldrich) was slowly added into a 90 ml hot solution (83° C.) of 100 mM potassium chloride (KCl) for 1 hr under magnetic stirring. Next, 0.2% (v/v) of span®80 (Sigma Aldrich) was added to 450 ml of paraffin oil (Sigma Aldrich) and heated to 83° C. After 1 hr, the Kcar solution was added to the oil phase and stirred under an overhead stirrer at 1500 RPM for 10 min. Then, the emulsion was allowed to cool to room temperature under continuous overhead stirring at 600 RPM. After cooling, the solution was transferred into 50 ml falcons and centrifuge at 3000 RCF for 5 min to separate the water and oil phases. The supernatant of the oil phase was removed and replaced with 70% ethanol. The samples were vortexed vigorously to resuspend the microgels and were centrifuged at 3000 RCF for 5 min. This step was repeated 3 times until most of the paraffin oil removed. Next, the sedimented microgels were suspended in 100 Mm KCl solution and centrifuged at 3000 RCF for 5 min. This wash step was repeated two times to remove the remaining ethanol. Next, to avoid aggregates, the samples were suspended with 100 mM KCl and filtered using Buchner funnel (60 μm pore nylon filter, Merck). At this stage the filtered samples can be stored in 4° C. for later use.

Prior to printing, the microgels were suspended with the relevant cross-linking solution according to the bioink: 0.1% (w/v) calcium chloride (CaCl2), 1000 [U/ml] thrombin (Evicel) in 1XPBS or 1XPBS for alginate, fibrin and GelMA, respectively. Next, the samples were vortexed vigorously and centrifuged at 3000 RCF for 5 minutes, than the supernatant was removed and the microgels were centrifuged again at 3000 RCF for 2 minutes to remove the remaining fluid. Lastly, measured volumes of the KCar microgels were transferred into 35 mm culture dishes or 24 well plates using a positive displacement pipette (Microman, Gilson).

For cell printing, the filtered samples were sterilized 3 times with 70% ethanol and centrifuged at 3000 RCF for 5 minutes. Next, the samples were washed twice with sterile 100 mM KCl and centrifuged at 3000 RCF for 5 minutes. Then, two washes were performed using sterile 1XPBS and DMEM low glucose, respectively and the samples were centrifuged at 3000 RCF for 5 minutes. The last wash before cell printing was performed using the cross-linking solution for the fibrin bioink: 1000 [U/ml] thrombin (Evicel) in DMEM low glucose and centrifuged at 3000 RCF for 5 minutes. The supernatant was removed and the sample was centrifuged again at 3000 RCF for 2 minutes to remove the remaining fluid.

### Size Distribution Measurements

The size distribution of KCar microgels was measured using a Malvern Mastersizer 2000. The microgel samples were diluted in 100 mM KCl and vortexed vigorously to obtain a homogenous dispersion before the measurements. The refractive indexes of KCar microgels and 100 Mm KCl solution were determined as 1.5 and 1.49 respectively.

### Structure Characterization Using Brightfield and Cryogenic Scanning Electron Microscopy (Cryo-SEM)

A dilute sample of KCar microgels were visualized using an inverted phase-contrast microscope. For cryogenic scanning electron microscopy (cryo-SEM) imaging a Zeiss Ultra Plus high-resolution SEM, equipped with a Schottky field-emission gun and with a BalTec VCT100 cold-stage maintained below −145° C. was used. Specimens were imaged at low acceleration voltages of 1 kV, and working distances of 3-5 mm. The Everhart Thornley (“SE2”) secondary electron imaging detectors were used. Low-dose imaging was applied to the specimen to minimize radiation damages.

Specimens were prepared by the drop plunging method, a 3 μL drop of solution is set on top of a special planchette maintaining its droplet shape and is manually plunged into liquid ethane, after which it is set on top of a specialized sample table. The frozen droplets are transferred into the BAF060 freeze fracture system, where they are fractured by a rapid stroke from a cooled knife, exposing the inner part of the drop. They are then transferred into the pre-cooled HR-SEM as described above. Ideally, imaging is performed as close as possible to the drop surface, where cooling rate should be maximal.

### Rheological Measurements

The rheological measurements were preformed using MCR 302 rheometer (Anton paar) with parallel plate geometry (25 mm diameter) and 1 mm gap. All samples (n=3) were centrifuged at 3000 RCF for 5 min and the supernatant was removed. Then, the samples were centrifuged again at 3000 RCF for 2 min to remove the remaining fluid. Next, 600 μl of the microgels was loaded on a rheometer plate for the measurements.

To examine the linear viscoelastic region (LVR) amplitude sweep experiments were performed. The storage (G′) and loss (G″) modulus were measured in the range of 0.01%-100% strain, under oscillation frequency of temperature sweep experiments were performed to examine the temperature sensitivity of the microgels. The storage (G′) and loss (G″) modulus were measured at a wide range of temperatures 20° C.-70° C. under 0.1% amplitude strain which is the value within the linear viscoelastic region (LVR).

Steady shear flow measurements were used in order to study the shear thinning behavior of the microgels dispersion. The viscosity was measured in a wide range of shear rates 0.001-100 [1/sec].

Thixotropic measurements were performed to investigate the recovery properties of the granular hydrogel. The storage (G′) and loss (G″) modulus were measured at low strain percentage of 0.1% (4 cycles) and high strain percentage of 100% (3 cycles).

### FRAP Analysis

Fluorescence Recovery after photobleachnig (FRAP) was performed using LSM700 confocal microscope. 0.1% (w/v) of fluorescein sodium salt (376 MW, Sigma Aldrich) was dissolved in 1XPBS. Samples of KCar microgels, gelatin support material or 1XPBS were prepared and mixed with fluorescein tracer solution in 1:1 ratio. The samples were vortex and 100 μl from each sample was transferred into 48-well plates. The FRAP experiment were performed by bleaching a circular area with a diameter of 150 μm for 50 iterations. The diffusion coefficients were calculated using open-source MATLAB code “frap analysis” (paper—A Method Improving the Accuracy of Fluorescence Recovery after Photobleaching Analysis).

### Transparency Test

To demonstrate and quantify the transparency of the granular hydrogel, absorbance measurements were performed using Synergy™ HTBioTek® (BioTek Instruments, Winooski, Vt., USA) at wavelengths of 400-700 nm. The results were compared to a turbid support material, Life Support (FluidForm). The samples were prepared and centrifuged according to the manufacturer protocol and transferred into 24-well plate for the measurements. The transmittance was calculated using the logarithmic relation and representative images of the materials demonstrate in FIG. 2.

### Bioink Preparation

2% (w/v) alginate (medium viscosity, sigma Aldrich) was dispersed in DDW with 0.1% (w/v) Alcian blue (alfa aesar) for visualization. The solution was stirred for overnight using magnetic stirring. GelMA bioink was prepared by dispersing 10% (w/v) of GelMA stock in 1Xphosphate buffer saline (PBS) at 37° C. overnight. 0.2% lithium phenyl-2,4,6-trimethylbenzoylphosphinate (LAP, Sigma-Aldrich) and 1.6% polyethylene oxide (PEO) were dispersed in 1XPBS at 37° C. overnight. Then to obtain a 5% (w/v) GelMA bioink the GelMA and the LAP-PEO solutions were mixed in a 1:1 ratio and store at 4° C. 15 min prior printing.

For experiments including cells, fibrin bioink was prepared by dissolving 3 mg/ml hyaluronic acid in DMEM low glucose medium supplemented with 1% pen/strep and 30 KIU/ml aprotinin. For experiments without cells, 3 mg/ml hyaluronic acid is dissolved in DMEM low glucose medium supplemented with 10% glycerol. Next, 30 mg/ml type. A gelatin is added and mixed at 37° C. Finally, 10 mg/ml fibrinogen is added and allowed to dissolve. The solution is then sterile filtered and stored at −20 C until use. Prior to use, the bioink is thawed at 37° C., and cells are suspended in the bioink at a concentration of 6 million cells per 1 ml of bioink.

Before use, bioinks (without cells) centrifuged for 20 seconds at 3000 g to remove air bubbles. Where necessary, 0.5 um fluorescent microspheres (Polysciences) are mixed at 1:100 ratio with the bioink to aid with fluorescent imaging.

### 3D Printing of Rectilinear Patterns and Large Constructs

Prepared bioinks are transferred into 3 cc printing cartridges (Nordson EFD) fitted with 300 μm inner diameter blunt end needle (CML supply). The printing cartridge is then loaded into a printing tool of 6 axis extrusion bioprinter BioAssemblyBot (Advanced Solutions).

The design of the printed constructs is done using TSIM (Advanced Solutions) or SolidWorks (Dassault Systêmes SolidWorks Corp., USA). The object is sliced to the desired slice thickness (either 80% or 50% of the needle inner diameter), and the file is sent to the bioprinter.

Before printing, the support material was centrifuge and suspend with the cross-linking solution as describe above. Finally, the sedimented microgels transferred into 35 mm culture dishes or 24 well plates. The printing is performed with specified pressure and speed parameters as determined by volumetric calibration of the printing parameters for each bioink. The volumetric calibration is performed by extruding bioink at a specified pressure for a set amount of time and measuring the extruded volume. Based on the principal of volume conservation, and the following equation, the optimal printing speed is calculated:

$$Volume ( droplet ) = Volume ( filament ) = {π ( \frac{d}{2} )}^{2} \cdot speed \cdot time$$

After printing, the constructs are allowed to fully crosslink for 30 minutes, followed by either medium addition, or construct extraction by gentle PBS pipetting and dilution of the support material.

### Cell Culture

Cells are thawed and seeded on 75 or 150 cm² cell culture flasks (Techno Plastic Products AG, Switzerland). All cell culture is performed in humidified incubators at 37° C., and in an HEPA-filtered atmosphere of air and 5% of CO₂. Medium changes are performed every 2-3 days, except where indicated otherwise.

Human neonatal dermal fibroblasts (HNDFs; Lonza Wakersville Inc., USA), or red fluorescent protein-expressing HNDF (HNDF-RFP; Angio-Proteomie, USA) are cultured in endothelial cell medium (ScienceCell) supplemented with 5% FBS (ScienceCell) and endothelial cell growth bullet kit (ScienceCell).

Mesenchymal stem cells (MSC; Lonza) are cultured in Nutristem medium (Biological Industries). Cells are harvested for experiments during passages 3-6 at approximately 70% of confluence. For live cell tracking of MSCs cells were labelled with either DiI, DiD or DiO Vybrant cell labelling solution (Thermo Fisher).

Propagation of hiPSCs and Directed Cardiac Monolayer Differentiation

In this study, hiPSC expressing GCaMP (kindly gifted by Prof. Bruce Conklin, Gladstone Institute, USA) were used and cultured on Matrigel coated plates with mTeSR-1 medium (StemCell Technologies). hiPSC were passaged by dissociation with 0.5 mM ethylenediaminetetraacetic acid (EDTA, Gibco) every 4-5 days. Differentiation of hiPSCs to cardiomyocytes was based on a previously described protocol. Briefly, when cells reached 80-90% confluence, the culture-medium was exchanged with a differentiation medium composed of RPMI-1640, 2% B27 supplement minus insulin (ThermoFisher Scientific), 1% penicillin/streptomycin, and supplemented with 6 μM CHIR99021 (Stemgent) for two days. Medium was then exchanged into RPMI/B27 supplemented with 2 μM Wnt-059 (Selleck Chemicals) for another two days. After the fifth day, cells were cultured with RPMI/B27 medium alone. Cardiomyocytes (after 10-14 days of differentiation) were enzymatically dissociated using TrypLE express (Gibco) into small clusters or single cardiomyocytes. For bioprinting experiments, 8 million cardiomyocytes were suspended in 1 ml fibrin bioink.

Optical Imaging GCaMP-Expressing hiPSC-CMs

To measure the fluorescence intensity of the GCaMP expressing cells within the printed tissue, the line-scan mode of a Zeiss LSM700 laser-scanning confocal microscope (Zeiss) was used. 1 μM isoproterenol (Sigma-Aldrich) was dissolved in water and added to the tissue solution. Recording of the calcium transients was performed 10 min after the addition of the pharmacological agent. The GCaMP fluorescent recordings were analyzed to characterize the properties of the optical calcium transients using the Clampfit 10.7 program (Molecular Devices). The evaluated parameters include the beating rate and the decay time of the calcium transient (defined as the time interval from the peak).

### Immunofluorescent Staining

Whole scaffolds are fixated in 4% paraformaldehyde (PFA; Electron Microscopy Sciences, USA) for 20 min, and then washed thrice with PBS, 5 minutes each wash. Next, the scaffolds are treated with 0.3% Triton X-100 (Bio Lab Ltd) for 10 min in order to permeabilize the cell membrane. The scaffolds are washed with PBS and soaked in blocking solution (10% w/v bovine serum albumin solution in PBS) overnight at 4° C. Afterwards, samples are incubated overnight at 4° C. with the following primary antibodies, diluted in blocking. After 3 washes, secondary antibodies diluted in PBS are added and incubated with the sample for 3 h at room temperature: Cy3-conjugated donkey anti-mouse IgG (1:100, Jackson Immuno-research laboratory, PA), and Alexa 488-conjugated donkey anti-rabbit IgG (1:800, Life Technologies). DAPI (1:1000; Sigma-Aldrich), is added to the secondary antibody solution, for nuclear counterstaining. The scaffolds are then washed thrice and stored at 4° C. in PBS, until imaging.

### Construct Imaging and Biological Parameters Quantification

The constructs are imaged using LSM700 confocal microscope or Axiovert 7 (Zeiss, Germany). Image analysis is done using Zen software (Zeiss, Germany) or FIJI ImageJ.

### Viability Assays

LIVE/DEAD Cell Viability Assay (Invitrogen) was performed according to the manufacturer's instructions. Briefly, 7 days following incubation, bioprinted constructs were washed once with PBS followed by incubation with Calcein AM and Ethidium homodimer-1 for 45 minutes at 37 C. The constructs were then imaged using Zeiss LSM700 confocal microscope. The images were analyzed using FIJI ImageJ and the number of live and dead cells was counted and the percentage of live cells was calculated.

The PrestoBlue® Cell Viability assay was performed by incubating the constructs 7 days following printing with the PrestoBlue reagent for 60 minutes. The absorbance vas measured using a plate reader and the percent reduction of PrestoBlue was calculated.

### Example 1

### Preparation and Characterization of Kcar Microgels

The main design criteria for a support material that can be utilized in “print-and-grow” approach is stability under culturing conditions for prolong times. Obviously, biocompatibility and lack of cytotoxicity is also required. Other key factors include clarity that enables live cell imaging, and easy and controllable fabrication.

Based on these considerations were chose to design and investigate a new support material composed of kappa-carrageenan (Kcar) microgels that were fabricated using water in oil (W/O) emulsion. Microgels are polymeric microscale particles with high water content and porosity. KCar is a biocompatible polysaccharide originating from red seaweeds, widely used in the food industry as thickening agent (Fontes-Candia et al., 2020). Furthermore, KCar is a thermosensitive polymer that undergoes gelation during cooling or in the presence of monovalent cations. The gelation process involves helix formation followed by further aggregations to form a strong transparent hydrogel. As described below, this property was utilized to fabricate stable KCar microgels without using any toxic crosslinking agents. In one embodiment, toxic crosslinking agent comprises potassium persulfate. In one embodiment, toxic crosslinking agent are well known in the art.

The first goal was to fabricate spherical particles in a replicable procedure; hence a method based on first creating a W/O emulsion at high temperature, followed by cooling the emulsion to induce gelation of KCar and separation of the microgels was developed. More specifically, an aqueous precursor solution containing KCar and ionic cross linker (potassium chloride, KCl) was mixed with an oil phase at high temperature in which the polymer is soluble, and stirred to generate water droplets within the continuous oil phase. The polymeric water droplets were stabilized using span®80 as surfactant. Upon cooling to room temperature the hydrogels cures and solidifies, which allowed it separation using centrifugation. The spherical morphology of the obtained microgels is visible in bright field microscopy images (FIG. 2A) and cryo-SEM micrographs (FIG. 2B). The size distribution of the microgels (average from 6 different preparations) indicated that small particles with number average diameter of 21.4 μm±2.1 μm were obtained (FIG. 2C). It is noted that by changes of the fabrication conditions it is possible to obtain microgels with different sizes.

First, strain sweep experiment was used in order to explore the linear viscoelastic region (LVR) of the granular hydrogel (FIG. 2D). This data demonstrates the transition from gel behavior (G′>G″) to fluid behavior (G″>G′) due to high shear strains. Next, the thermo sensitive behavior of the granular hydrogel through temperature sweep experiments (FIG. 2E) where the storage (G′) and Loss (G″) moduli were measured in response to temperature increase was examined. The results revealed gel-like behavior (G′>G″) in all tested temperatures, particularly at ambient temperature and at physiological temperature. This behavior enables continuous mechanical support of solid particles during printing and cellular construct incubation. The shear-thinning behavior was studied using steady shear flow measurements (FIG. 2f). The viscosity was measured at increasing shear rates to simulate the needle movement through the granular hydrogel. The granular support exhibits a decrease in viscosity with an increase of shear rate which was well fitted to simple power low model derived from the Carreau model: η=k×\dot{γ}ⁿ⁻¹ Where η is the measured viscosity, \dot{γ} is the shear rate, k is the consistency coefficient and n is the power low index or flow behavior index. The power law index depends on the shear thinning characteristics of a fluid; n<1 for shear thinning materials whereas n>1 for shear thickening materials. The qualitative observation shows that the viscosity decreases with shear rate, the calculated parameters (n=0.21±0.01, k=(8.26±0.74)×10⁴ [mPa·sec]) suggest shear thinning behavior of the granular hydrogel. Finally, thixotropy measurements were performed to characterize the self-healing and recovery properties. The experiments were performed by using cycles of small amplitude oscillations (γ=0.1%) to test the initial microgels properties. Then, large amplitude oscillations (γ=100%) were performed to destroy the packed microgels structure and test the recovery to the initial properties (FIG. 2G). The results of the thixotropy measurement detects a gel-like behavior under low shear strains (G′>G″) and a liquid-like behavior under high shear strains (G″>G′). Furthermore, rapid and full recovery was demonstrated in all cycles indicating the self-healing properties of the granular hydrogel.

In order to grow cellular constructs within the support material, it is necessary to ensure nutrients diffusion. Therefore, the diffusion of a small molecule through the support material using fluorescence recovery after photo bleaching (FRAP) was tested. The diffusion coefficient of fluorescein tracer (376 Da) through the granular hydrogel was calculated and compared to the diffusion coefficient of the same molecule in commercial support bath composed of gelatin particles (LifeSupport) and 1× phosphate buffer used as controls (FIG. 21). The diffusion coefficient through the KCar microgels was significantly higher (p<XXX) than in the gelatin support bath, demonstrating potential for faster nutrients transport. These results demonstrate molecular transport through the void spaces, although the high packing density of the microgels.

In addition to the morphology, porosity and rheological properties, an important feature of the granular hydrogel is its transparency. The printing process involves multiple parameters that affect the printability of low viscosity biomaterials, therefore visualization of the printed structure through the support material can ease and improve the calibration of the printer and the bioink. To quantify the transparency of the microgels, the absorbance of light passing through the granular hydrogel and the transmittance spectra was calculated and compared to a commercial support material (Life support). The average transmittance (400-700 nm) of the granular hydrogel (55%) was higher than the gelatin particles (23%) and less than liquid buffer (92%). The transparency of the support materials is demonstrated in FIG. 2G.

### Example 2

### Printing Resolution and Large Constructs

A versatile support material needs to support various types of extruded bioinks with different gelation mechanisms. Here, three widely used bioinks were tested: Alginate with calcium chloride as ionic cross linker, gelatin-methacryloyl (GelMA) that undergoes photo-cross linking and fibrin which undergoes enzymatic cross-linking. In order to verify the bioink printability within the KCar support and quantify shape fidelity, a planar structure of 4 layers rectilinear pattern with an internal square shape (2 mm×2 mm) was printed. To aid the visualization of the extruded bioinks, either alcian blue powder or 0.5 μm fluorescent microspheres were added to the bioink (FIG. 3A). Then, a semi-quantitative evaluation of the printed structures using the printability index (Pr) to quantify geometric accuracy within the granular hydrogel (FIG. 3D) was performed. The results display high geometric accuracy compared to the designed model and lack of significant difference between the bioinks, demonstrating the versatility of the granular hydrogel.

Biological scaffolds are typically 3D bulk objects with complex architecture; therefore, the printing of volumetric large-scale construct within the granular hydrogel was examined. First, the printing accuracy in x, y, z direction by printing the “Technion” university logo, using the above bioinks (FIG. 3C) was demonstrated. Next, it was aimed to print a vascular structure due to the critical role of nutrition and oxygen transport in cellular scaffolds. A hollow diverging vessel was printed using the same bioinks and extracted from the granular hydrogel using gentle pipetting (FIG. 3B). To demonstrate the open lumen of the structure a red food dye was perfused through the channels (movieS1). The hollow vessels were perfusable and robust, validating the high resolution and fidelity of the constructs. Moreover, the resulting structures had sufficient structural integrity to be extracted from the granular hydrogel and handled freely.

### Example 3

### Contraction of Cell Laden Hydrogel

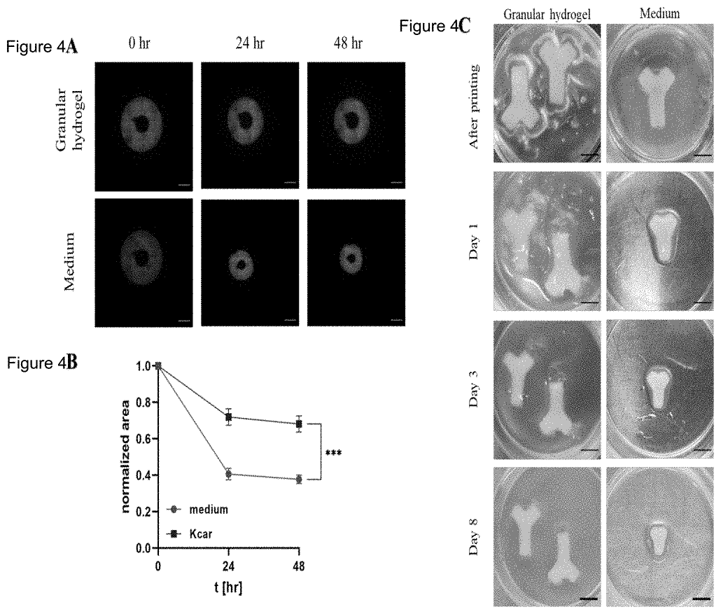

Cell laden hydrogels are known to contract during the culture period following fabrication. To investigate the effect of incubation within the granular hydrogel on the shrinkage of printed cellular scaffolds, either labelled-MSCs or labelled-HNDFs encapsulated in a fibrin-based bioink were printed. The scaffolds were printed in a concentric disc shape with an outer and inner diameter of 7 mm and 3 mm respectively. The scaffolds were subsequently cultured within the support material. Scaffolds extracted from the granular support immediately after printing and transferred to liquid medium served as a control. The cells were immediately imaged after printing, 24 hours and 48 hours after printing (FIG. 4A) and measured the inner and outer diameters. The total area of the constructs was calculated and normalized to the initial area after printing (FIG. 4B). The results show that control scaffolds that were extracted to medium shrink, and their total area was reduced to 37% for HNDFs and 15-45% for MSCs 48 hours after printing. On the other hand, the scaffolds incubated in KCar support medium exhibited significantly reduced contraction and their total area was reduced to 68% and 20-80% for HNDFs and MSCs respectively. To further investigate the effect of the mechanical support on scaffold contraction during culture, labelled-HNDFs were printed in a diverging blood vessel structure. The printed vessels were either cultured within the support material or extracted to a liquid medium after printing. The scaffold contraction was examined during culture for 8 days and images were taken using light (FIG. 4C) or fluorescence microscope (figure S). The scaffold contraction in the liquid medium was more pronounced than the contraction within the granular hydrogel, hence the support medium enhances the dimensional stability of the construct. This results further emphasize the potential of the granular hydrogel to support the integrity of cellular scaffolds during culture.

### Example 4

### Cell Viability

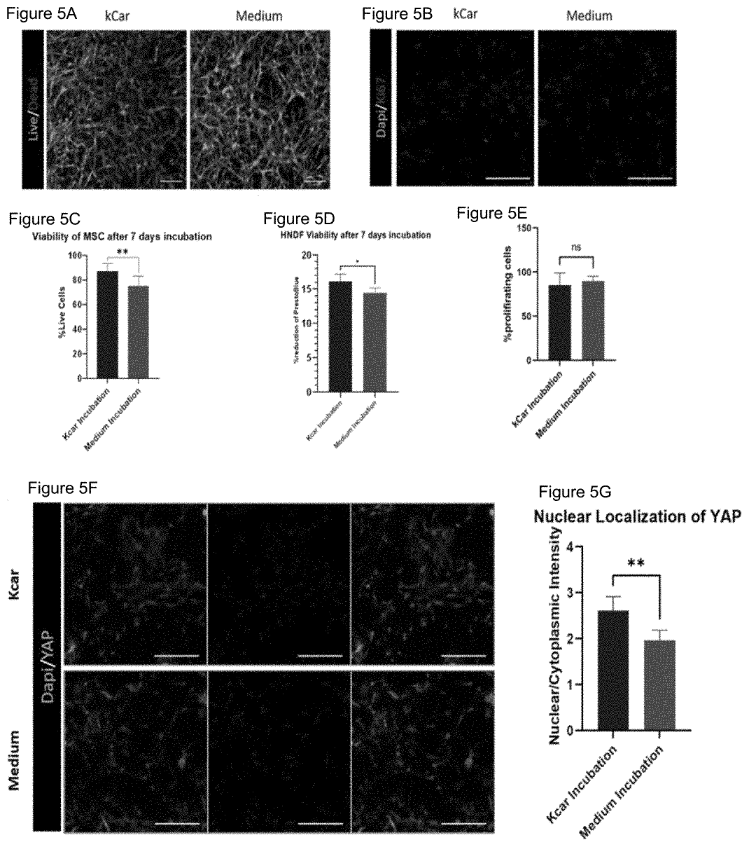

Printed cellular constructs incubated within support material need to maintain their viability. To examine the viability of cells, a live/dead staining assay on MSCs that were printed and incubated within KCar granular hydrogel for 7 days (FIG. 5A) was performed. It was found that cells incubated in the granular hydrogel had an average percentage of live cells of 87.1%, while the control groups extracted from the support material had an average percentage of live cells of 75.2% (FIG. 5C). Moreover, a colorimetric Presto Blue viability assay for HNDFs printed constructs and incubated in the granular hydrogel for 7 days was performed. The results show that HNDFs incubated in the granular hydrogel had a higher reduction of presto blue, indicative of a higher number of viable cells as compared to a control group extracted after printing. Lastly, in order to verify that these viable cells are proliferating, fixed cryo-sections of printed HNDFs for proliferation marker ki67 were stained. Quantification of ki67 positive cells yielded a similar percentage of proliferating cells in constructs that were incubated in the granular hydrogel or extracted from it (FIG. 5E).

In addition, it was hypothesized that the microgel swelling after incubation with medium can apply mechanical forces on the printed scaffolds. In order to examine this effect, cryo-sections of printed HNDFs in fibrin gels for Yes associated protein (YAP) expression (FIG. 5F) were stained. A higher nuclear localization of YAP in constructs that were incubated in the granular hydrogel as compared to those that were extracted (FIG. 5G) was observed. These results demonstrate the viability of the cells within the granular hydrogel during culture and thus confirms the usefulness of the print and grow cellular scaffolds of the present invention.

### Example 5

### Cell Function

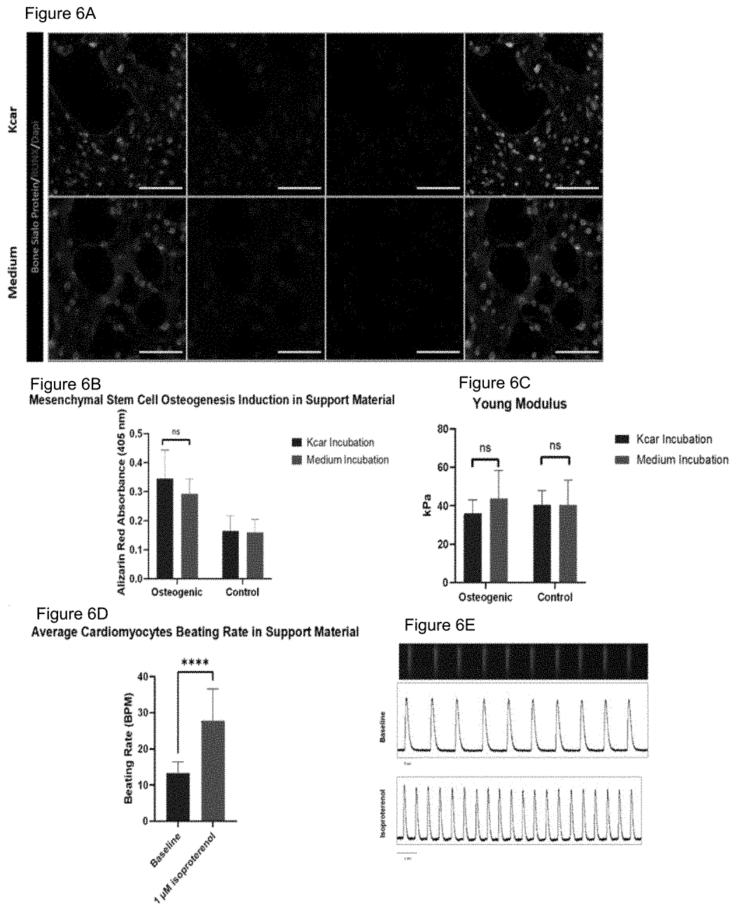

In order to verify that cells can function properly when incubated inside the granular hydrogel, MSC-laden fibrin bioink constructs were printed and incubated inside the granular hydrogel. Constructs extracted to liquid medium served as controls. After printing, the medium of the cells was exchanged for osteogenic medium to drive the differentiation of MSCs into osteoblasts. The constructs were examined for osteogenic markers, as well as measured their mechanical stiffness. Immunofluorescent staining for nuclear early osteogenic marker RUNX and late cytoplasmic osteogenic marker BSP-2 show similar expression in both groups (FIG. 6A). Similar alizarin red assay (FIG. 6B) and Young modulus (FIG. 6C) results also suggest that incubation in the granular hydrogel does not negatively impact the mechanical stiffness or the mineral deposition of differentiating MSCs. These results indicate that the capability of MSC to differentiate into osteoblasts is preserved after incubation in the support material for 7 days.

Lastly, the function of iPSC-derived cardiomyocytes expressing GCaMP incubated in the support material was assessed. Cardiomyocytes were suspended in fibrin bioink and printed in a wave pattern. After 4 days of incubation inside the support material, the constructs were imaged and observed the characteristic rhythmic calcium flux and contraction. Moreover, it was aimed to examine the cells response to drugs that can alter the beat rate. Isoproterenol was added to the medium and recorded the fluorescent signal associated with the calcium flux. The cells responded to the drug, and the beat rate increased. These results show that incubation in the support material enables the generation of functional and viable cellular constructs that maintain their shape.

1. A kappa-carrageenan (Kcar) granular hydrogel devoid of a cell-toxic crosslinking agent.

2. A water in oil emulsion comprising the kappa-carrageenan (Kcar) granular hydrogel of claim 1.

3. The kappa-carrageenan (Kcar) granular hydrogel of claim 1, composed of 0.001 to 0.05% (w/v) Kappa carrageenan and 85-98% (w/v) water.

4. A composition comprising the water in oil emulsion of claim 2, a lipophilic non-ionic surfactant, and an ionic cross linker.

5. A composition comprising a kappa-carrageenan (Kcar) granular hydrogel of claim 1 and potassium chloride.

6. The composition of claim 5, comprising a lipophilic non-ionic surfactant.

7. A composition comprising a kappa-carrageenan (Kcar) granular hydrogel of claim 1 and a bioink.

8. A particle comprising a kappa-carrageenan (Kcar) granular hydrogel of claim 1, characterized by a diameter of 1 micrometer to 100 micrometers.

9. A composition comprising a kappa-carrageenan (Kcar) granular hydrogel of claim 1, encapsulating a cell.

10. A composition comprising a kappa-carrageenan (Kcar) granular hydrogel of claim 1, and at least one compound selected from: calcium chloride, thrombin, fibrin, gelatin-methacryloyl, or any combination thereof.

11. A composition comprising a kappa-carrageenan (Kcar) granular hydrogel of claim 1, and cell culture media.

12. A composition comprising a kappa-carrageenan (Kcar) granular hydrogel of claim 1, wherein said hydrogel comprises a lumen within its matrix.

13. The composition of claim 12, wherein said lumen is having the structure and dimensions of a blood vessel.

14. The composition of claim 12, wherein said lumen is defined or surrounded by cells.

15. A method for fabricating a cell carrier, comprising inserting a cell into a composition comprising the kappa-carrageenan (Kcar) granular hydrogel of claim 1.

16. A method for implanting a cell to a subject, comprising administering to said subject the composition of claim 9.
